(19) United States
(12) Patent Application Publication
(10) Pub. No.: US 2019/0302134 A1
(43) Pub. Date: Oct. 3, 2019

(54) DETERMINING MELATONIN LEVEL IN A BIOLOGICAL SAMPLE

(71) Applicant: KONINKLIJKE PHILIPS N.V., EINDHOVEN (NL)

(72) Inventors: LAURENT BROUQUEYRE, MARIETTA, GA (US); MANUEL LAURA LAPOINT, PITTSBURGH, PA (US); ALEXANDER VAN REENEN, VUGHT (NL); MARKUS HENDRIKUS VAN ROOSMALEN, BERKEL-ENSCOT (NL)

(21) Appl. No.: 16/359,333

(22) Filed: Mar. 20, 2019

Related U.S. Application Data
(60) Provisional application No. 62/649,822, filed on Mar. 29, 2018.

(51) Int. Cl. G01N 33/94

(52) U.S. Cl. CPC G01N 33/9406; G01N 2800/2864

(57) ABSTRACT

The disclosure pertains to determining a melatonin level in a biological sample of a subject with a determination system. The determination system comprises a melatonin analyzer, a temperature sensor, and a controller. The method comprises providing a first biological sample from the subject to the melatonin analyzer; generating one or more output signals conveying information related to a temperature of the analyzer; controlling the temperature of the analyzer based on the output signals to be within a pre-determined temperature range, such that responsive to the temperature of the analyzer being outside the pre-determined temperature range, the controlling comprises cooling and/or heating the analyzer to bring and maintain the temperature within the pre-determined temperature range; and responsive to the temperature of the analyzer being within the pre-determined temperature range, facilitating determination of a melatonin level of the first biological sample with the analyzer.

### CROSS-REFERENCE TO RELATED APPLICATIONS

This patent application claims the priority benefit under 35 U.S.C. § 119(e) of U.S. Provisional Application No. 62/649,822 filed on Mar. 29, 2018, the contents of which are herein incorporated by reference.

### BACKGROUND

### 1. Field

The present disclosure pertains to a system and method for determining a melatonin level in a biological sample of a subject.

### 2. Description of the Related Art

Determining melatonin levels in biological samples of subjects is typically used to determine dim light melatonin onset (DLMO) in the subject. Determining DLMO may be helpful in analyzing or diagnosing various diseases and circadian disorders, such as circadian rhythm sleep disorders (e.g., insomnia), jet lag, seasonal affective disorder, shift work-related de-synchronies, and the delayed sleep phase syndrome. Current methods for determining DLMO require measuring the actual concentrations of melatonin in biological samples (e.g., blood, saliva, urine, or other bodily fluid).

Current methods include taking a predefined number of samples from an individual, each of the samples being taken at a different time interval, and sending the samples to laboratories that have access to melatonin immuno-assays. It usually takes two to three weeks for the requester to receive their result. Further, to ensure that a sufficient number of samples are sent for analysis, the predefined number of samples taken is often greater than the actual number of samples needed to determine DLMO, thereby resulting in greater inconvenience to the individual. The present disclosure overcomes at least these deficiencies in prior art systems.

### SUMMARY

Accordingly, one or more aspects of the present disclosure relate to a method for determining a melatonin level in a biological sample of a subject with a determination system, the determination system comprising a melatonin analyzer, a temperature sensor, and a controller. The method comprises providing a first biological sample from the subject to the melatonin analyzer; generating, with the temperature sensor, one or more output signals conveying information related to a temperature of the analyzer; controlling, with the controller, the temperature of the analyzer based on the output signals to be within a pre-determined temperature range, such that responsive to the temperature of the analyzer being outside the pre-determined temperature range, the controlling comprises cooling and/or heating the analyzer to bring and maintain the temperature within the pre-determined temperature range; and responsive to the temperature of the analyzer being within the pre-determined temperature range, facilitating, with the controller, determination of a melatonin level of the first biological sample with the analyzer.

Another aspect of the present disclosure relates to a system configured for determining a melatonin level in a biological sample of a subject. The system comprises a melatonin analyzer configured to receive a first biological sample from the subject. The system comprises a temperature sensor configured to generate one or more output signals conveying information related to a temperature of the analyzer. The system comprises a controller configured to: control the temperature of the analyzer to be within a pre-determined temperature range based on the output signals, such that responsive to the temperature of the analyzer being outside the pre-determined temperature, the controller is configured to cool and/or heat the analyzer to bring and maintain the temperature within the pre-determined temperature range; and facilitate determination of a melatonin level of the first biological sample responsive to the temperature of the analyzer being within the pre-determined temperature range.

Still another aspect of the present disclosure relates to a system configured for determining a melatonin level in a biological sample of a subject. The system comprises means for analyzing configured to receive a first biological sample from the subject. The system comprises means for generating one or more output signals conveying information related to a temperature of the means for analyzing. The system comprises means for controlling the temperature of the means for analyzing based on the output signals to be within a pre-determined temperature range, such that responsive to the temperature of the means for analyzing being outside the pre-determined temperature range, the controlling comprises cooling and/or heating the means for analyzing to bring and maintain the temperature within the pre-determined temperature range. The system comprises means for facilitating determination of a melatonin level of the first biological sample responsive to the temperature of the means for analyzing being within the pre-determined temperature range.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a system configured for determining a melatonin level in a biological sample of a subject, in accordance with one or more embodiments;

FIG. 2 illustrates example of a system configured for determining a melatonin level in a biological sample of a subject, in accordance with one or more embodiments; and

FIG. 3 illustrates example operations performed by a system configured for determining a melatonin level in a biological sample of a subject, in accordance with one or more embodiments.

### DETAILED DESCRIPTION OF EXEMPLARY EMBODIMENTS

As used herein, the singular form of “a,” “an,” and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

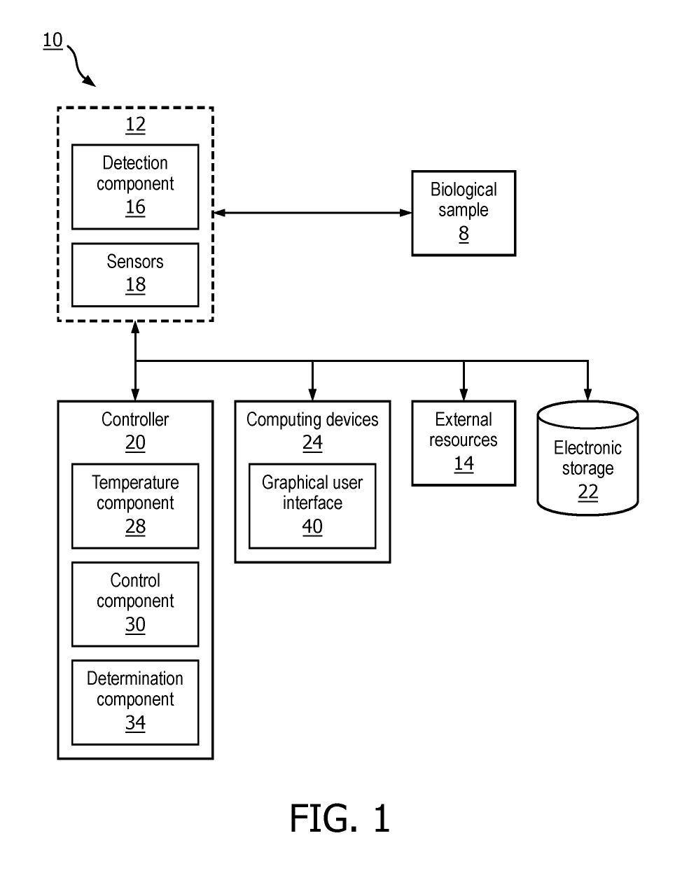

FIG. 1 is a schematic illustration of a system 10 configured for determining a melatonin level in a biological sample of a subject, in accordance with one or more embodiments. In prior art systems, biological sample taking for melatonin level determination starts in the beginning of the evening before the subject begins to sleep, usually 2 hours before normal bedtime. Samples are usually taken once per hour and are refrigerated once they are collected. Five or six samples are taken and are expected to cover the person's melatonin secretion onset time. To provide economies of scale, a typical research lab or sleep center which collects the samples usually ships the samples to a specialized laboratory for analysis once they have collected enough samples to justify a refrigerated shipment. To provide economies of scale, the processing laboratory processes the melatonin measurements once they have received enough samples to compose a full batch. The turnaround for receiving the results is usually several weeks long. DLMO may be determined only after this long processing period.

System 10 is configured to facilitate determining DLMO overnight (real-time measurements) and/or in other timeframes, thereby cutting the wait time compared to prior art systems significantly. In some embodiments, the biological samples are processed based on a melatonin determination response time (assay response time) as described herein. In some embodiments, the melatonin determination response time may vary based on the intrinsic assay kinetics and on the processing temperature control. System 10 is configured to control temperature of the reactions (of the assays) in a range that is not currently used for this type of reaction (e.g., above freezing, and below body temperature). In some embodiments, at least because of this temperature control, system 10 facilitates processing the biological samples within a sampling time of one hour or even less. In some embodiments, the samples are processed within a sampling time of 30 minutes, 20 minutes and or other intervals. In some embodiments, system 10 may allow for determining DLMO of multiple subjects overnight. For example, for economies of scale (cost saving gained by an increased level of production) the relationship between the sampling time and the speed of analysis may be an integer, so that a complete number of samples can be analyzed while samples are being taken, which allows for more patients to be analyzed per analyzer. For example if the reaction time can be decreased to 30 minutes, two patients' profiles can be measured using only one analyzer; if the reaction time can be decreased to 20 minutes, 3 patients' profiles can be produced overnight; etc.

In some embodiments, system 10 comprises an analyzer 12, a controller 20, electronic storage 22, client computing device(s) 24, external resources 14, and/or other components. In FIG. 1, analyzer 12, controller 20, electronic storage 22, external resources 14, and client computing device(s) 24 are shown as separate entities. In some embodiments, some or all of the components of system 10 and/or other components may be grouped into one or more singular devices (e.g., a medical device, a diagnosis device, a therapy device, a wearable device, or other user devices). In some embodiments, system 10 may not include one or more of the components shown in FIG. 1 (e.g., controller 20), and the operations performed by those components may be performed by other components in system 10, and other systems and/or devices.

In some embodiments, analyzer 12 is configured to receive one or more biological samples 8 from a subject. In some embodiments, analyzer 12 is configured to detect one or more analytes in the biological sample 8 of the subject. In some embodiments, the one or more analytes may include melatonin, adrenaline, insulin, aldosterone, antidiuretic hormone, oxytocin, prolactin, ghrelin, leptin, cortisone, cortisol, blood levels of sugar or cholesterol, or other substance/constituent, in the biological samples 8 of the subject. In some embodiments, the biological samples 8 may be blood, saliva, urine, or other bodily fluid. In some embodiments, analyzer 12 is configured to detect melatonin levels in the biological sample 8 with an immunoassay. A melatonin immunoassay is any method for detecting a melatonin by using an antibody reactive with melatonin. In some embodiments, analyzer 12 may be configured to determine/detect analyte (e.g., melatonin) levels in the biological sample using techniques such as enzyme-linked immunosorbent assays (ELISA) or liquid chromatography-mass spectroscopy (LC-MS). ELISA is an analytic biochemistry assay that uses a solid-phase enzyme immunoassay (ETA) to detect the presence of a substance. LC-MS is an analytical chemistry technique that combines the physical separation capabilities of liquid chromatography with the mass analysis capabilities of mass spectrometry. The type of assay presented herein is not intended to be limiting, as any suitable technique that provides analyte level determination may be used.

In some embodiments, analyzer 12 may include detection component 16, a cartridge component 11, sensor(s) 18, and /or other components. In some embodiments, detection component 16 is configured to receive the biological sample for incubation with an antibody (e.g., a melatonin antibody). In some embodiments, detection component 16 comprise a melatonin sensor configured to generate one or more signals conveying information related to the melatonin level in the biological sample. In some embodiments, detection component 16 may be a sensor surface. In some embodiments, detection component 16 may include one or more light sensors (e.g., photoresistor-based sensors, photodiode-based sensors, phototransistor-based sensors, etc.), sound sensors (e.g., microphones), chemical sensors (e.g., sensors that include a chemical (molecular) recognition system (receptor), a physicochemical transducer, etc.), or other sensors. In some embodiments, detection component may include or be included in sensor(s) 18.

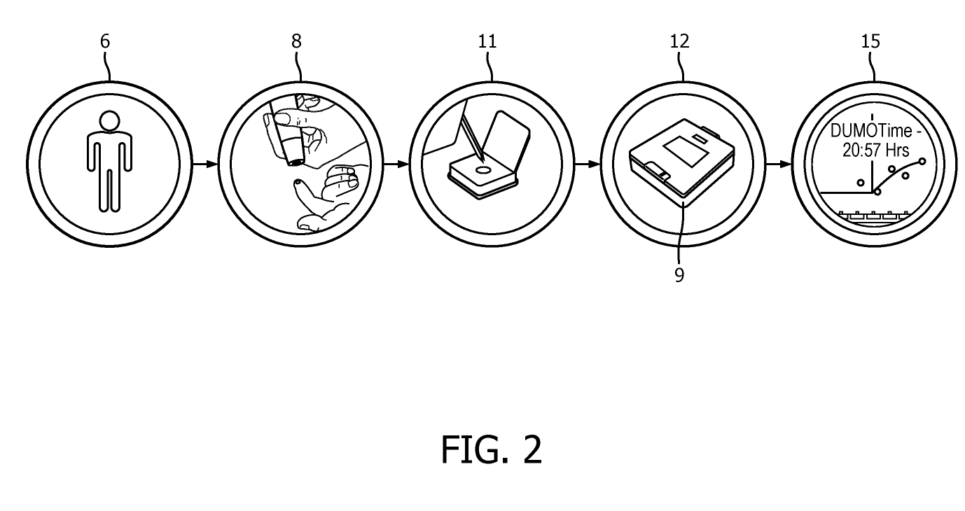

Cartridge component 11 (shown in FIG. 2) is configured to receive the biological sample. In some embodiments, cartridge component 11 may include detection component 16. In some embodiments, cartridge component 11 may be a surface, a microfluidic chamber, a reaction chamber, or a particle surface. In some embodiments, cartridge component 11 may be disposable. In some embodiments, in operation, a melatonin antibody may be printed on a surface in cartridge component 11. In this example, free melatonin in the biological sample is detected in the presence of manufactured melatonin coupled to magnetic or paramagnetic beads. In this example, melatonin from the biological sample binds to the surface of cartridge component 11 containing melatonin antibodies. Subsequently, magnetic particles coated with melatonin (not from the sample, e.g., manufactured) are brought in contact with the surface. Depending on the amount of melatonin from the biological sample that has bound to the surface, more or less magnetic particles will bind to the surface. In other words, melatonin from the sample inhibits the binding reaction of magnetic particles to the surface. This is just one method of detecting/determining melatonin level in the biological sample. Other methods may be used and are in accordance with the present techniques. Not excluding complex detection of melatonin captured by an antibody that is for example printed on the plastic surface.

In some embodiments, analyzer 12 may include a housing (9) (shown in FIG. 2), one or more sensors (e.g., sensor(s) 18), processors (e.g., controller 20), one or more analyte detection components (e.g., detection component 16), and/or other components within or outside system 10. In some embodiments, analyzer 12 may be included in a medical device (e.g., a device similar to the Minicare device by Philips™). In some embodiments, one or more components of analyzer 12 may be located within or outside the housing of analyzer 12. For example, in some embodiments, analyzer 12 may be configured to include one or more sensors, one or more controllers, one or more cartridges, and one or more analyte detection components within the housing. In some embodiments, analyzer 12 may be configured to include one or more sensors, one or more cartridges, and one or more analyte detection components within the housing and one or more controllers outside of the housing. In some embodiments, analyzer 12 may be configured to include one or more controllers and one or more analyte detection components, and one or more cartridges within the housing and one or more sensors outside of the housing. Such sensors, controllers, analyte detection components, and other components of analyzer 12, whether housed within or outside of the housing, may communicate with one another via wired or wireless connections. It should be noted that, although some embodiments are described herein with respect to an analyzer 12 performing certain operations, one or more such operations may be performed by one or more other components (e.g., one or more servers, client devices, etc.). As an example, such other components (e.g., one or more servers, client devices, etc.) may include one or more processor components that are the same as or similar to components 30-34 described below.

Sensor(s) 18 is configured to generate one or more output signals conveying information related to a temperature of analyzer 12. In some embodiments, sensor(s) 18 may be configured to output information related to temperature of the assay, the cartridge, and/or the housing. In some embodiments, sensor(s) 18 may be a temperature sensor (e.g., a thermocouple, a thermistor, an infrared sensor, a thermometer, a resistive temperature measuring devices (RTD), and or other temperature sensors). In some embodiments, sensor(s) 18 may be located in cartridge component 11. In some embodiment, sensor(s) 18 may include one or more light sensors (e.g., photoresistor-based sensors, photodiode-based sensors, phototransistor-based sensors, etc.), sound sensors (e.g., microphones), chemical sensors (e.g., sensors that include a chemical (molecular) recognition system (receptor), a physicochemical transducer, etc.), or other sensors. Although sensor(s) 18 is illustrated at a single location in system 10, this is not intended to be limiting. Sensor(s) 18 may include sensors disposed in a plurality of locations within system 10.

Controller 20 is configured to control operations of one or more components of system 10. In some embodiments, controller 20 may include one or more processors configured to provide information processing capabilities in system 10. As such, controller 20 may include one or more of a digital processor, an analog processor, and a digital circuit designed to process information, an analog circuit designed to process information, a state machine, and/or other mechanisms for electronically processing information. Although controller 20 is shown in FIG. 1 as a single entity, this is for illustrative purposes only. In some embodiments, controller 20 may include a plurality of processing units. These processing units may be physically located within the same device (e.g., a server), or controller 20 may represent processing functionality of a plurality of devices operating in coordination (e.g., one or more servers, one or more computing devices 24 associated with users, a diagnostic device, a medical device, a therapy device, analyzer 12, detection component 16, sensor(s) 18, a piece of a hospital equipment, devices that are part of external resources 14, electronic storage 22, and/or other devices.)

As shown in FIG. 1, controller 20 is configured to execute one or more computer program components. The one or more computer program components may comprise a temperature component 28, a control component 30, a determination component 34, and/or other components. Controller 20 may be configured to execute components 28, 30, 34, and/or other components by software; hardware; firmware; some combination of software, hardware, and/or firmware; and/or other mechanisms for configuring processing capabilities on controller 20.

It should be appreciated that although components 28, 30, and 34, are illustrated in FIG. 1 as being co-located within a single processing unit, in embodiments in which controller 20 comprises multiple processing units, one or more of components 28, 30, 34, and/or other components may be located remotely from the other components. The description of the functionality provided by the different components 28, 30, 34, and/or other components described below is for illustrative purposes, and is not intended to be limiting, as any of components 28, 30, and/or 34 may provide more or less functionality than is described. For example, one or more of components 28, 30, and/or 34 may be eliminated, and some or all of its functionality may be provided by other components 28, 30, and/or 34. As another example, controller 20 may be configured to execute one or more additional components that may perform some or all of the functionality attributed below to one of components 28, 30, and/or 34.

Temperature component 28 is configured to receive output signals form sensor(s) 18 that convey information related to temperature of analyzer 12. In some embodiments, temperature component 28 is configured to determine temperature of analyzer 12 based on the received output signals. In some embodiments, temperature component 28 may be configured to determine temperature of an assay in analyzer 12, one or more cartridges within analyzer 12, detection component 16, and/or other components of analyzer 12 based on the received output signals form sensor(s) 18. In some embodiment, temperature component 28 may be configured to output visual information including temperature measurements (e.g., of analyzer 12, an assay in analyzer 12, one or more cartridges within analyzer 12, detection component 16, and/or other components of analyzer 12) via user interface 40 (described below). In some embodiments, the output may be in the form of a graphical, digital, textual, and/or other representation format of the sound parameters.

In some embodiments, temperature component 28 may be configured to record the temperature of analyzer 12 or other component of analyzer 12 (e.g., in temperature component 28, storage 22, external resources 14, and/or in other components within or outside system 10). In some embodiments, temperature component 28 may be configured to record the determined temperature for a predetermined period of time (e.g., duration of an assay). In some embodiments, temperature component 28 may be configured to continuously determine/record temperature of the analyzer (and/or other components of the analyzer). In some embodiments, temperature component 28 may be configured to periodically determine/record the temperature.

Control component 30 is configured to control one or more operations of one or more components of system 10. In some embodiments, control component 30 may be configured to control operations of analyzer 12. In some embodiments, control component 30 may be configured to start, pause, and/or stop operations of analyzer 12 or operations of other components of system 10. In some embodiments, control component 30 may control operations of analyzer 12 (or operations of other components of system 10) based on a user selection (e.g., via user interface 24, and/or one or more other buttons, controls, etc.). In some embodiments, control component 30 may control operations of analyzer 12 (or operations of other components of system 10) based on information from other components within or outside system 10. In some embodiments, control component 30 may control operations of analyzer 12 (or operations of other components of system 10) based on output signals received from sensor(s) 18. For example, in some embodiments, control component 30 may start, pause or stop operations of analyzer 12 in response to receiving temperature information form temperature component 28 (or from sensor (s) 18 or other components of system 10).

In some embodiments, control component 30 is configured to control the temperature of analyzer 12 based on the output signals and/or the temperature determined by temperature component 28. In some embodiments, control component 30 controls temperature of analyzer 12 (or other components of analyzer 12) to be within a pre-determined temperature range (e.g., an upper and lower temperature thresholds). In some embodiments, the pre-determined temperature range of the analyzer (or other component of analyzer 12) may be above freezing and below room temperature. In some embodiments, the pre-determined temperature range of the analyzer (or other component of analyzer 12) may be above freezing and below body temperature. For example, the pre-determined temperature range of the analyzer may be between about 0 and about 30 degrees Celsius. In some embodiments, the pre-determined temperature range of the analyzer may be between about 19 and about 21-23 degrees Celsius. In some embodiments, the controller may control the temperature of analyzer 12 to be 20 degrees Celsius.

In some embodiments, control component 30 is configured to control cooling and/or heating analyzer 12 to bring and maintain the temperature within the pre-determined temperature range. For example, controller 20 may be configured to control operations of a cooling and/or heating element (within analyzer 12, or system 10) to cool and/or heat analyzer 10 to bring and maintain temperature of the analyzer to the predetermined range. In some embodiments, system 10 may include one or more cooling and/or heating elements located within or outside analyzer 12. In some embodiments, for example, system 10 may include a heater configured to control temperature of analyzer 12 at 20 degrees Celsius. Analyzer 12 and/or system 10 may be (or may be placed) in a low temperature environment (e.g., about 10 degrees Celsius). In this example, the heater may be configured to bring and maintain temperature of analyzer to about 20 degrees Celsius. In some embodiments, for example, system 10 may include a cold plate located within analyzer 12 (or outside analyzer 12) and configured to cool analyzer 12 (and/or other components of analyzer 12) such that the temperature is maintained at the predetermined range (e.g., below room temperature). In some embodiments system 10 may comprise a refrigerator configured to control the temperature of analyzer 10 to be within the predetermined range. For example, analyzer 12 may be placed in the refrigerator such that the assay reaction are performed in the temperature-controlled environment of the refrigerator. In some embodiments, system 10 may include a Peltier element configured to cool and/or heat analyzer 10 to control the temperature of analyzer 12. In some embodiments, control component 30 may be configured to control an air conditioner, a heater, and/or other environmental temperature control devices that control the temperature of the ambient environment around analyzer 12.

In some embodiments, in operation, temperature of analyzer 12 is determined based on the output signals received from sensor(s) 18. The determined temperature is compared to one or more pre-determined temperatures (e.g., temperature range stored in an electronic storage within or outside system 10, or provided by the user) such that responsive to the temperature of analyzer 12 being outside the pre-determined temperature range, the controller controls operations of the cooling and/or heating elements to bring and maintain the temperature within the pre-determined temperature range (by cooling or heating analyzer 12). In some embodiments, the operations of determining the temperature of analyzer and controlling the temperature to be within a pre-determined range may be accomplished before or after the biological sample is provided to analyzer 12. In some embodiments, these operations are part of a pre-calibration of analyzer 12. The pre calibration involves measurement of samples with known melatonin concentrations and the use of this information the make standard curves to calculate unknown samples melatonin concentrations.

Determination component 34 is configured to determine an analyte level of the first biological sample (e.g., melatonin level). In some embodiments, component 34 is configured to determine the melatonin level of the sample responsive to the temperature of the analyzer being within the pre-determined temperature range (e.g., above freezing and below room temperature). Making this determination while the temperature of analyzer 12 is in the pre-determined temperature range cuts the response time of determining the melatonin level of the biological sample to less than one hour, for example. In some embodiments, the response time is less than 30 minutes. In some embodiments, the response time is less than 20 minutes.

In some embodiments, component 34 is configured to determine analyte levels (e.g., melatonin) of subsequent biological samples within a pre-determined sampling time (responsive to the temperature of the analyzer being within the pre-determined temperature range). The sampling time may be pre-determined. In some embodiment, the pre-determined sampling time is based on the response time of determining analyte (e.g., melatonin) levels of the previous biological sample. For example if the response time of determining the previous sample is one hour, the sampling time is at least one hour; if the response time of determining the previous sample is 30 minutes, the sampling time is at least 30 minutes; if the response time of determining the previous sample is 20 minutes, the sampling time is at least 20 minutes; etc.

For example, in operation, a first sample may be provided to the analyzer for melatonin level detection. A subsequent sample may be provided to the analyzer after a first melatonin level in the first sample have been determined (responsive to the temperature being within the predetermined range as explained above). A subsequent sample may be provided to the analyzer at an interval that corresponds to the response time of detecting melatonin level of the sample that precedes it. For example, the sampling time may be within one hour, within 30 minutes, within 20 minutes, or other intervals.

In some embodiments, determination component 34 is configured to determine dim light melatonin onset (DLMO) of the subject based on the determined melatonin level of the biological samples of the subject (e.g., of the first biological sample and the subsequent melatonin levels of the subsequent biological samples). The melatonin levels of unknown samples are calculated based on predefined calibrations curves determined upon manufacturing of the cartridges. Using the calculated melatonin levels a dose time curve is built and used to calculate the moment of significant increase of melatonin levels within the patient. Alternatively, the constant low melatonin level is established and significant increase of melatonin is calculated compared to the determined low melatonin levels. This can be done on raw signals, i.e. without use of standard curves, or using a predetermined calibration curve and calculation of melatonin levels. In case of raw signals the baseline response level (first samples) are used to determine the moments of significant decrease in signal levels as the moment of DLMO. In some embodiments, determination component 34 may be configured for determining DLMO of the subject overnight. In some embodiments, the biological samples are processed based on the melatonin determination response time (assay response time). In some embodiments, the samples are processed within a sampling time of 30 minutes, 20 minutes and/or other intervals. In some embodiments, determination component 34 may be configured for determining DLMO of multiple subjects overnight. For example, a complete number of samples can be analyzed while samples are being taken, which allows for more patients to be analyzed per analyzer. For example if the reaction time can be decreased to 30 minutes, two patients' profiles can be measured using only one analyzer; if the reaction time can be decreased to 20 minutes, 3 patients' profiles can be produced overnight; etc.

FIG. 2 illustrates example of a system configured for determining a melatonin level in a biological sample of a subject, in accordance with one or more embodiments. The system comprises a melatonin analyzer 12 configured to receive biological samples 8 from the subject 6. The system comprises a cartridge component 11 for receiving the biological samples. Cartridge 11 may be included in analyzer 12. In some embodiments, cartridge component 11 is configured to be received in analyzer 12. In some embodiments, analyzer 12 comprises housing (9) configured to receive (or house) cartridge component 11. In some embodiments, the system comprises a temperature sensor (not shown) configured to generate one or more output signals conveying information related to temperature of analyzer 12. In some embodiments, the temperature sensor may be located within or outside analyzer 12. The system comprises a controller (not shown) configured to control the temperature of analyzer 12 to be within a pre-determined temperature range based on the measured temperature of analyzer 12. For example, the controller is configured to cool and/or heat the analyzer to bring and maintain the temperature within the pre-determined temperature range responsive to the temperature of the analyzer being outside the pre-determined temperature. The system is configured to facilitate determination of the melatonin level of the biological sample responsive to the temperature of the analyzer being within the pre-determined temperature range. In some embodiments, subsequent biological samples 8 may be processed to determine melatonin levels in the subsequent samples 8. A DLMO 15 of the subject may be determined based on the melatonin levels of the biological samples 8

Returning to FIG. 1, external resources 14 include sources of subject information and/or other information. In some embodiments, external resources 14 include sources of subject information and/or other information, such as databases, websites, etc., external entities participating with system 10 (e.g., a medical records system of a healthcare provider that stores medical history information for populations of subjects), one or more servers outside of system 10, a network (e.g., the internet), electronic storage, equipment related to Wi-Fi technology, equipment related to Bluetooth® technology, data entry devices, sensors, scanners, and/or other resources. In some embodiments, some or all of the functionality attributed herein to external resources 14 may be provided by resources included in system 10. External resources 14 may be configured to communicate with controller 20, computing devices 24, electronic storage 22, and/or other components of system 10 via wired and/or wireless connections, via a network (e.g., a local area network and/or the internet), via cellular technology, via Wi-Fi technology, and/or via other resources.

Electronic storage 22 includes electronic storage media that electronically stores information. The electronic storage media of electronic storage 22 may include one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 22 may be (in whole or in part) a separate component within system 10, or electronic storage 22 may be provided (in whole or in part) integrally with one or more other components of system 10 (e.g., computing devices 24, controller 20, etc.). In some embodiments, electronic storage 22 may be located in a server together with controller 20, in a server that is part of external resources 14, in a computing device 24, and/or in other locations. Electronic storage 22 may include one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 22 may store software algorithms, information determined by controller 20, information received via a computing device 24 and/or graphical user interface 40 and/or other external computing systems, information received from external resources 14, analyzer 12, sensor(s) 18, and/or other information that enables system 10 to function as described herein.

Computing devices 24 are configured to provide interfaces between caregivers (e.g., doctors, nurses, friends, family members, etc.), subjects, and/or other users, and system 10. This enables data, cues, results, instructions, recommendations, and/or any other communicable items, collectively referred to as “information,” to be communicated between a user (e.g., the subject, a doctor, a caregiver, and/or other users) and analyzer 12, controller 20, electronic storage 22, and/or other components of system 10. For example, client computing device(s) 24 may display a representation of the output signal from sensor(s) 18 (e.g., visual signals, raw sound signals, graphical signals, digital signals, numeric data, video, audio, text, etc.) to a user. In some embodiments, client computing device(s) 24 comprises at least one interface that is provided integrally with controller 20, analyzer 12, sensor(s) 18, and/or other components of system 10. In some embodiments, individual computing devices 24 may be included, in desktop computers, laptop computers, tablet computers, smartphones, and/or other computing devices associated with individual caregivers, subjects, and/or other users. In some embodiments, individual computing devices 24 are, and/or are included, in equipment used in hospitals, doctor's offices, home testing environments, and/or other medical equipment; test equipment; equipment for treating subjects; therapy equipment; data entry equipment; and/or other devices.

Computing devices 24 are configured to provide information to, and/or receive information from, the caregivers, subjects, and/or other users. In some embodiments, computing devices 24 are configured to present a graphical user interface 40 to the user to facilitate display representations of the temperature of analyzer 12, the analyte levels, data analysis of analyte levels, and/or other information. In some embodiments, the output may be in the form of a graphical, digital, textual, and/or other representation format of the sound parameters. In some embodiments, graphical user interface 40 includes a plurality of separate interfaces associated with computing devices 24, controller 20 and/or other components of system 10; multiple views and/or fields configured to convey information to and/or receive information from caregivers, subjects, and/or other users; and/or other interfaces.

In some embodiments, computing devices 24 are configured to provide graphical user interface 40, processing capabilities, databases, and/or electronic storage to system 10. As such, computing devices 24 may include controllers 20, electronic storage 22, external resources 14, and/or other components of system 10. In some embodiments, computing devices 24 are connected to a network (e.g., the internet). In some embodiments, computing devices 24 do not include controllers 20, electronic storage 22, external resources 14, and/or other components of system 10, but instead communicate with these components via the network. The connection to the network may be wireless or wired. For example, controller 20 may be located in a remote server and may wirelessly cause display of graphical user interface 40 to the caregivers on computing devices 24. As described above, in some embodiments, an individual computing device 24 is a laptop, a personal computer, a smartphone, a tablet computer, and/or other computing devices. Examples of interface devices suitable for inclusion in an individual computing device 24 include a touch screen, a keypad, touch-sensitive and/or physical buttons, switches, a keyboard, knobs, levers, a display, speakers, a microphone, an indicator light, an audible alarm, a printer, and/or other interface devices. The present disclosure also contemplates that an individual computing device 18 includes a removable storage interface. In this example, information may be loaded into a computing device 24 from removable storage (e.g., a smart card, a flash drive, a removable disk, etc.) that enables the caregivers, subjects, and/or other users to customize the implementation of computing devices 24. Other exemplary input devices and techniques adapted for use with computing devices 24 include, but are not limited to, an RS-232 port, an RF link, an IR link, a modem (telephone, cable, etc.), and/or other devices.

In some embodiments, all or some component of system 10 may be communicatively coupled via a network. The network may include the Internet and/or other networks, such as local area networks, cellular networks, Intranets, near field communication, frequency (RF) link, Bluetooth™, Wi-Fi™, and/or any type(s) of wired or wireless network(s). Such examples are not intended to be limiting, and the scope of this disclosure includes embodiments in which analyzer 12, external resources 14, sensor(s) 18, controller(s) 20, electronic storage 22, and/or client computing device(s) 24 are operatively linked via some other communication media.

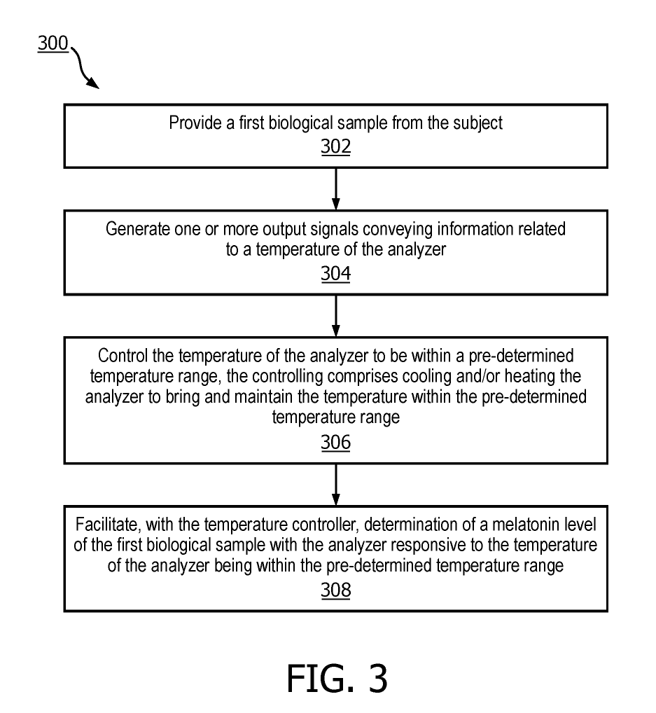

FIG. 3 illustrates a method 300 for determining a melatonin level in a biological sample of a subject with a determination system. The system comprises an analyzer, a temperature sensor, a controller, and/or other components. The operations of method 300 presented below are intended to be illustrative. In some embodiments, method 300 may be accomplished with one or more additional operations not described, and/or without one or more of the operations discussed. Additionally, the order in which the operations of method 300 are illustrated in FIG. 3 and described below is not intended to be limiting.

In some embodiments, method 300 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 300 in response to instructions stored electronically on an electronic storage medium. The one or more processing devices may include one or more devices configured through hardware, firmware, and/or software to be specifically designed for execution of one or more of the operations of method 300.

At an operation 302, a first biological sample from the subject is provided to the analyzer. In some embodiments, operation 302 is performed by one or more melatonin analyzer the same as or similar to analyzer 12 (shown in FIG. 1 and described herein).

At an operation 304, output signals conveying information related to temperature of the analyzer are generated. In some embodiments, operation 304 is performed by one or more sensors the same as or similar to sensor(s) 18 (shown in FIG. 1 and described herein).

At an operation 306, the temperature of the analyzer is controlled based on the output signals to be within a pre-determined temperature range. In some embodiments, the temperature of the analyzer is controlled such that responsive to the temperature of the analyzer being outside the pre-determined temperature range, the controlling comprises cooling and/or heating the analyzer to bring and maintain the temperature within the pre-determined temperature range. In some embodiments, operation 306 is performed by a controller the same as or similar to controller 20 (shown in FIG. 1 and described herein).

At an operation 308, determination of a melatonin level of the first biological sample is facilitated. In some embodiments, determination of the melatonin level of the first biological sample is facilitated responsive to the temperature of the analyzer being within the pre-determined temperature range. In some embodiments, operation 308 is performed by a controller the same as or similar to controller 20 (shown in FIG. 1 and described herein).

In some embodiments, operations of system 10 may be accomplished without one or more components. For example, in some embodiments, operations of system 10 may be accomplished without a temperature sensor and a controller. For example, in some embodiments, the temperature sensor may be outside of system 10 (e.g., the temperature sensor may be a sensor that measures temperature of the location where system 10 is located). In some embodiments, control of temperature of the analyzer (or system 10, or the assay) may be accomplished by controlling temperature of the location where system 10 is located.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

What is claimed is:

1. A method for determining a melatonin level in a biological sample of a subject with a determination system, the determination system comprising a melatonin analyzer, a temperature sensor, and a controller, the method comprising:
providing a first biological sample from the subject to the melatonin analyzer;
generating, with the temperature sensor, one or more output signals conveying information related to a temperature of the analyzer;
controlling, with the controller, the temperature of the analyzer based on the output signals to be within a pre-determined temperature range, such that responsive to the temperature of the analyzer being outside the pre-determined temperature range, the controlling comprises cooling and/or heating the analyzer to bring and maintain the temperature within the pre-determined temperature range; and
being responsive to the temperature of the analyzer being within the pre-determined temperature range, facilitating, with the controller, determination of a melatonin level of the first biological sample with the melatonin analyzer.

2. The method of claim 1, wherein the pre-determined temperature range of the melatonin analyzer is above freezing and below body temperature, such that a response time of determining the melatonin level of the first biological sample is less than one hour.

3. The method of claim 1, wherein the pre-determined temperature range of the analyzer is above freezing and below room temperature.

4. The method of claim 1, wherein the pre-determined temperature range of the analyzer is between 19 and 21 degrees Celsius.

5. The method of claim 1, further comprising:
providing subsequent biological samples from the subject to the analyzer within a pre-determined sampling time, the pre-determined sampling time being determined based on a response time for determining the melatonin level of the first biological sample;
being responsive to the temperature of the analyzer being within the pre-determined temperature range, facilitating, with the controller, determination of subsequent melatonin levels of the subsequent biological samples with the analyzer; and
determining dim light melatonin onset (DLMO) of the subject based on the determined melatonin level of the first biological sample and the subsequent melatonin levels of the subsequent biological samples.

6. The method of claim 5, wherein the predetermined sampling time is between 20 and 30 minutes.

7. The method of claim 1, wherein the melatonin analyzer comprises a housing, a cartridge and a melatonin detection component, and wherein the method further comprises detecting with the melatonin detection component melatonin in the biological sample based on a reaction, in the cartridge, between the melatonin antibody and the melatonin in the biological sample.

8. The method of claim 7, wherein the cartridge is one or more of a microfluidic chamber, a reaction chamber, and/or a particle surface.

9. The method of claim 7, wherein the melatonin detection component is a melatonin sensor configured to generate one or more signals conveying information related to the melatonin level in the first biological sample.

10. A system configured for determining a melatonin level in a biological sample of a subject, the system comprising:
a melatonin analyzer configured to receive a first biological sample from the subject;
a temperature sensor configured to generate one or more output signals conveying information related to a temperature of the analyzer; and
a controller configured to:
control the temperature of the analyzer to be within a pre-determined temperature range based on the output signals, such that responsive to the temperature of the analyzer being outside the pre-determined temperature, the controller is configured to cool and/or heat the analyzer to bring and maintain the temperature within the pre-determined temperature range; and
facilitate determination of a melatonin level of the first biological sample responsive to the temperature of the analyzer being within the pre-determined temperature range.

11. The system of claim 10, wherein the controller is configured to control the temperature of the analyzer to be above freezing and below body temperature, such that a response time of determining the melatonin level of the first biological sample is less than one hour.

12. The system of claim 10, wherein the controller is configured to control the temperature of the analyzer to be above freezing and below room temperature.

13. The system of claim 10, wherein the controller is configured to control the temperature of the analyzer to be between 19 and 21 degrees Celsius.

14. The system of claim 10, wherein
the melatonin analyzer is configured to receive subsequent biological samples from the subject to the melatonin analyzer within a pre-determined sampling time, the pre-determined sampling time being determined based on a response time for determining the melatonin level of the first biological sample; and
the controller is configured to:
being responsive to the temperature of the analyzer being within the pre-determined temperature range, facilitating determination of subsequent melatonin levels of the subsequent biological samples; and
determining dim light melatonin onset (DLMO) of the subject based on the determined melatonin level of the first biological sample and the subsequent melatonin levels of the subsequent biological samples.

15. The system of claim 14, wherein the predetermined sampling time is between 20 and 30 minutes.

16. The system of claim 10, wherein the analyzer comprises:
a housing;
a cartridge configured to receive the biological sample and a melatonin antibody; and
a detection component configured to detect melatonin in the biological sample based on a reaction between the melatonin antibody and the melatonin in the biological sample.

17. The system of claim 16, wherein the cartridge is one or more of a microfluidic chamber, a reaction chamber, and/or a particle surface.

18. The system of claim 16, wherein the melatonin detection component is a melatonin sensor configured to generate one or more signals conveying information related to the melatonin level in the first biological sample.
